(19) United States
(12) Patent Application Publication
(10) Pub. No.: US 2022/0223265 A1
(43) Pub. Date: Jul. 14, 2022

(54) MEDICAL PERSONNEL INFORMATION MANAGEMENT METHOD, MEDICAL PERSONNEL INFORMATION MANAGEMENT DEVICE AND NON-TRANSITORY MEMORY COMPUTER-READABLE STORAGE MEDIUM

(71) Applicants: Beijing Zhongxiangying Technology Co., Ltd., Beijing (CN); BOE Technology Group Co., Ltd., Beijing (CN)

(72) Inventors: Chuanqi HUANG, Beijing (CN); Wanwan TANG, Beijing (CN); Sichen WANG, Beijing (CN)

(21) Appl. No.: 17/556,754

(22) Filed: Dec. 20, 2021

(30) Foreign Application Priority Data
Jan. 14, 2021 (CN) 202110051079.0

(51) Int. Cl. G16H 40/20

(52) U.S. Cl. CPC G16H 40/20

(57) ABSTRACT

A medical personnel information management method, a medical personnel information management device and a non-transitory memory computer-readable storage medium are provided. The method includes: when a selection instruction for at least one medical staff is received, determining at least one medical staff; displaying a scheduling data configuration interface in response to a received scheduling instruction; determining a to-be-scheduled date and whether a scheduling template is used for scheduling; when the scheduling template is determined to be used for scheduling, calling the scheduling template to perform schedule each medical personnel to obtain scheduling data; when the scheduling template is determined not to be used for scheduling, in response to a scheduling data configuration instruction, configuring a plurality of to-be-scheduled time periods and an acceptable number of visitors associated with each to-be-scheduled time period in the to-be-scheduled date, to obtain the scheduling data.

### CROSS-REFERENCE TO RELATED APPLICATIONS

This application claims priority to Chinese Patent Application No. 202110051079.0 filed in China on Jan. 14, 2021, the entire contents of which are incorporated herein by reference.

### TECHNICAL FIELD

The application relates to the technical field of information management, in particular to a medical personnel information management method, a medical personnel information management device and a non-transitory memory computer-readable storage medium.

### BACKGROUND

When the disease is released (particularly after the outbreak of new coronary epidemic situation), the hospital becomes a high-risk area, a large number of non-emergency outpatients of the hospital are closed, the patients cannot go to the hospital for further treatment in time, and the on-line inquiry becomes an important form for obtaining medical services for the patients during the epidemic prevention period.

### SUMMARY

A medical personnel information management method is provided in the present disclosure, including:

when a selection instruction for at least one medical staff is received, determining at least one medical staff;

displaying a scheduling data configuration interface in response to a received scheduling instruction;

determining a to-be-scheduled date and whether a scheduling template is used for scheduling;

when the scheduling template is determined to be used for scheduling, calling the scheduling template to perform schedule each medical personnel to obtain scheduling data, where the scheduling template includes to-be-scheduled time periods and an acceptable number of visitors associated with each to-be-scheduled time period in the to-be-scheduled date;

when the scheduling template is determined not to be used for scheduling, in response to a scheduling data configuration instruction, configuring a plurality of to-be-scheduled time periods and an acceptable number of visitors associated with each to-be-scheduled time period in the to-be-scheduled date for each medical personnel on the scheduling data configuration interface, to obtain the scheduling data.

Optionally, the scheduling template is configured by:

configuring at least one template name in a template configuration interface in response to a template configuration instruction; and

for each template name, configuring a plurality of to-be-scheduled time periods and the acceptable number of visitors associated with each to-be-scheduled time period, and storing the acceptable number of visitors.

Optionally, the medical personnel information management method further includes:

determining whether to perform a batch scheduling;

when the batch scheduling is determined to be performed, determining a plurality of target dates which take the to-be-scheduled dates as starting points and are within a preset batch scheduling date range, and applying the scheduling data to each target date.

Optionally, the determining the plurality of target dates which take the to-be-scheduled dates as the starting points and are within the preset batch scheduling date range includes:

acquiring at least one scheduling date strategy, determining a plurality of dates which accord with the batch scheduling date range and each scheduling date strategy by taking the to-be-scheduled dates as the starting points, and taking the dates as a plurality of target dates;

the at least one scheduling date strategy includes at least one of: date excepting weekends, date excepting holidays, each date in a specified range of consecutive dates reserved, odd dates reserved, even dates reserved.

Optionally, the medical personnel information management method further includes:

determining dimension information for displaying the scheduling data, and determining a date to be displayed;

acquiring the scheduling data of the medical personnel regularly according to the dimension information and the date to be displayed; and

displaying the corresponding scheduling data.

Optionally, the medical personnel information management method further includes:

determining and displaying a current scheduling occupancy rate according to the used scheduling data of the current date and all the scheduling data of the current date; the used scheduling data are data which are actually used before the current time in the scheduling data of the current date.

Optionally, the medical personnel information management method further includes:

determining whether the work of each medical personnel is overtime according to a time-position relation between a scheduling time period to which the used scheduling data of each medical personnel belongs and the current time; the used scheduling data is an actually used data before the current time in the scheduling data of the current date;

if the work of the medical staff is overtime, sending first early warning information to a display terminal of the medical staff.

Optionally, the medical personnel information management method further includes:

if the work of the medical staff is not overtime, predicting whether the work of the medical staff is overtime according to a proportional relation between the data belonging to the current scheduling time period in the used scheduling data and the scheduling data of the current scheduling time period;

if the working time of the medical staff is predicted to be overtime, sending second early warning information to the display terminal of the medical staff.

Optionally, the medical personnel information management method further includes:

in response to an information query instruction for the current scheduling time period, displaying at least one of reservation information, used scheduling data and to-be-used scheduling data of the current scheduling time period; the used scheduling data is actually used data before the current time in the scheduling data of the current date, and the to-be-used scheduling data is data except the used scheduling data in all the scheduling data of the current date.

Optionally, the medical personnel information management method further includes:

sending basic information of each medical staff to an information security system, and receiving a unique identification of each medical staff fed back by the information security system based on the basic information;

in response to an electronic signature calling request of any one of medical staff, calling an interface associated with the unique identification of the medical staff to obtain a requested electronic signature.

Optionally, the medical personnel information management method further includes:

in response to a login request of any medical staff for logging in a medical staff management system, displaying an authorization interface displaying whether the calling of electronic signature is allowed;

in response to an instruction of allowing the calling of electronic signature, allowing the login request and sending authorization information to the information security system.

Optionally, the in response to an electronic signature calling request of any one of medical staff, calling the interface associated with the unique identification of the medical staff to obtain the requested electronic signature includes:

when an electronic signature calling request of any medical personnel is received, sending the electronic signature calling request to the information security system;

in response to an instruction of allowing the calling of electronic signature fed back by the information security system, calling the interface associated with the unique identifier of the medical staff to acquire an electronic signature of the medical staff; the instruction of allowing the calling of electronic signature is generated by the information security system upon verifying that the electronic signature is authorized;

in response to a call rejection instruction fed back by the information security system, displaying an authorization interface for judging whether to authorize to call the electronic signature, in response to an authorization approval instruction, calling the interface associated with the unique identifier of the medical staff to acquire the electronic signature of the medical staff, and uploading authorization information to the information security system; the rejection call instruction is generated by the information security system when the electronic signature is verified to be unauthorized.

A medical personnel information management device is further provided in the present disclosure, including: a memory, a processor electrically connected to the memory, where a computer program is stored in the memory, and the processor is configured to execute the computer program to:

when a selection instruction for at least one medical personnel is received, determine at least one medical personnel;

display a scheduling data configuration interface in response to a received scheduling instruction;

determine a to-be-scheduled date and whether a scheduling template is used for scheduling;

when the scheduling template is determined to be used for scheduling, call the scheduling template to perform schedule each medical personnel to obtain scheduling data, where the scheduling template includes to-be-scheduled time periods and an acceptable number of visitors associated with each to-be-scheduled time period in the to-be-scheduled date;

when the scheduling template is determined not to be used for scheduling, in response to a scheduling data configuration instruction, configure a plurality of to-be-scheduled time periods and an acceptable number of visitors associated with each to-be-scheduled time period in the to-be-scheduled date for each medical personnel on the scheduling data configuration interface, to obtain the scheduling data.

Optionally, the scheduling template is configured by:

configuring at least one template name in a template configuration interface in response to a template configuration instruction; and

for each template name, configuring a plurality of to-be-scheduled time periods and the acceptable number of visitors associated with each to-be-scheduled time period, and storing the acceptable number of visitors.

Optionally, the processor is configured to execute the computer program to:

determine whether to perform a batch scheduling;

when the batch scheduling is determined to be performed, determine a plurality of target dates which take the to-be-scheduled dates as starting points and are within a preset batch scheduling date range, and apply the scheduling data to each target date.

Optionally, the processor is configured to execute the computer program to:

acquire at least one scheduling date strategy, determining a plurality of dates which accord with the batch scheduling date range and each scheduling date strategy by taking the to-be-scheduled dates as the starting points, and take the dates as a plurality of target dates;

the at least one scheduling date strategy includes at least one of: date excepting weekends, date excepting holidays, each date in a specified range of consecutive dates reserved, odd dates reserved, even dates reserved.

Optionally, the processor is configured to execute the computer program to:

determine dimension information for displaying the scheduling data, and determining a date to be displayed;

acquire the scheduling data of the medical personnel regularly according to the dimension information and the date to be displayed; and

display the corresponding scheduling data.

Optionally, the processor is configured to execute the computer program to:

determine and display a current scheduling occupancy rate according to the used scheduling data of the current date and all the scheduling data of the current date; the used scheduling data are data which are actually used before the current time in the scheduling data of the current date.

Optionally, the processor is configured to execute the computer program to:

determine whether the work of each medical personnel is overtime according to a time-position relation between a scheduling time period to which the used scheduling data of each medical personnel belongs and the current time; the used scheduling data is an actually used data before the current time in the scheduling data of the current date;

if the work of the medical personnel is overtime, send first early warning information to a display terminal of the medical personnel.

Optionally, the processor is configured to execute the computer program to:

if the work of the medical personnel is not overtime, predict whether the work of the medical personnel is overtime according to a proportional relation between the data belonging to the current scheduling time period in the used scheduling data and the scheduling data of the current scheduling time period;

if the working time of the medical personnel is predicted to be overtime, send second early warning information to the display terminal of the medical personnel.

A non-transitory memory computer-readable storage medium storing a computer program is provided in the present disclosure, where the computer program is executed by a processor to:

when a selection instruction for at least one medical personnel is received, determine at least one medical personnel;

display a scheduling data configuration interface in response to a received scheduling instruction;

determine a to-be-scheduled date and whether a scheduling template is used for scheduling;

when the scheduling template is determined to be used for scheduling, call the scheduling template to perform schedule each medical personnel to obtain scheduling data, where the scheduling template comprises to-be-scheduled time periods and an acceptable number of visitors associated with each to-be-scheduled time period in the to-be-scheduled date;

when the scheduling template is determined not to be used for scheduling, in response to a scheduling data configuration instruction, configure a plurality of to-be-scheduled time periods and an acceptable number of visitors associated with each to-be-scheduled time period in the to-be-scheduled date for each medical personnel on the scheduling data configuration interface, to obtain the scheduling data.

### BRIEF DESCRIPTION OF THE DRAWINGS

The above and/or additional aspects and advantages of the present disclosure will become apparent and readily appreciated from the following description of the embodiments, taken in conjunction with the accompanying drawings of which:

FIG. 1 is a schematic flowchart of a medical personnel information management method according to an embodiment of the present disclosure;

FIG. 2 is a schematic illustration of a display and operation interface for a list of medical personnels in an embodiment of the present disclosure;

FIG. 3 is a schematic view of a display and operation interface for a department list in an embodiment of the present disclosure;

FIG. 4 is a schematic diagram of a scheduling data configuration interface according to an embodiment of the present disclosure;

FIG. 5 is a schematic diagram of another scheduling data configuration interface in an embodiment of the present disclosure;

FIG. 6 is a schematic diagram of a template configuration interface in an embodiment of the present disclosure;

FIG. 7 is a schematic diagram of another scheduling data configuration interface according to an embodiment of the present disclosure;

FIG. 8 is an expanded flow diagram of an alternative implementation of a medical personnel information management method provided in an embodiment of the present disclosure;

FIG. 9 is a schematic flow chart diagram illustrating a portion of another medical personnel information management method according to an embodiment of the present disclosure;

FIG. 10 is a schematic view of a display interface for scheduling data according to the embodiment of the present disclosure;

FIG. 11 is a partial schematic flow chart diagram illustrating a medical personnel information management method according to an embodiment of the present disclosure;

FIG. 12 is a schematic flow chart of a portion of still another medical personnel information management method according to an embodiment of the present disclosure;

FIG. 13 is a schematic flowchart illustrating a portion of yet another medical personnel information management method according to an embodiment of the present disclosure;

FIG. 14 is a schematic diagram of information interaction between an Internet hospital system and a CA system in the embodiment of the present disclosure;

FIG. 15 is a schematic diagram of binding of a doctor's job number and a unique identifier in an embodiment of the present disclosure;

FIG. 16 is a schematic diagram of a dynamically generated interface call communication token and its associated token in an embodiment of the present disclosure;

FIG. 17 is a schematic diagram of an authorization interface of the present disclosure whether to authorize invocation of an electronic signature;

FIG. 18 is a schematic diagram of a prescription after an electronic signature is added in an embodiment of the present disclosure;

FIG. 19 is a schematic view of a prescription interface for a patient query in an embodiment of the present disclosure;

FIG. 20 is a schematic view of a prescription interface for physician query in an embodiment of the present disclosure;

FIG. 21 is a schematic structural framework diagram of a medical personnel information management device according to an embodiment of the present disclosure; and

FIG. 22 is a schematic structural framework diagram of a medical personnel information management device according to an embodiment of the present disclosure.

### DETAILED DESCRIPTION

The present disclosure is described in detail below and examples of embodiments of the present disclosure are illustrated in the accompanying drawings, where like reference numerals refer to the same or similar elements or elements with the same or similar functionality throughout. In addition, if a detailed description of the known art is unnecessary for the features of the present disclosure shown, it is omitted. The embodiments described below with reference to the accompanying drawings are exemplary only for explaining the present disclosure and are not construed as limiting the present disclosure.

It will be understood by those within the art that, unless otherwise defined, all terms (including technical and scientific terms) used herein have the same meaning as commonly understood by one of ordinary skill in the art to which this application belongs. It will be further understood that terms, such as those defined in commonly used dictionaries, should be interpreted as having a meaning that is consistent with their meaning in the context of the prior art and will not be interpreted in an idealized or overly formal sense unless expressly so defined herein.

As used herein, the singular forms “a”, “an”, “the” and “the” are intended to include the plural forms as well, unless the context clearly indicates otherwise. It will be further understood that the terms “includes” and/or “including,” when used in this specification, specify the presence of stated features, integers, steps, operations, elements, and/or components, but do not preclude the presence or addition of one or more other features, integers, steps, operations, elements, components, and/or groups thereof. It will be understood that when an element is referred to as being “connected” or “coupled” to another element, it can be directly connected or coupled to the other element or intervening elements may also be present. Further, “connected” or “coupled” as used herein may include wirelessly connected or wirelessly coupled. As used herein, the term “and/or” includes all or any element and all combinations of one or more of the associated listed items.

The inventor of the application finds through research that the existing hospital shift arrangement scheme has low efficiency and cannot meet the practical requirements, in addition, medical care personnel generally need to perform safety verification when calling information by a medical care personnel management system, the existing safety verification mode needs to log in through a U-key (a small storage device which is directly connected to a computer through a Universal Serial Bus (USB) interface, has a password verification function and is reliable and high-speed) for identity authentication so as to call corresponding information, and the mode has the following defects that 1) the U-key is easy to lose; 2) the U-key damage can cause data exception and cannot be used continuously. Therefore, the existing security verification mode is poor in convenience and affects user experience.

The application provides a medical personnel information management method, a medical personnel information management device, medical personnel information management equipment and a storage medium, and aims to solve the technical problems in the prior art.

The following describes the technical solution of the present disclosure and how to solve the above technical problems in detail by specific embodiments.

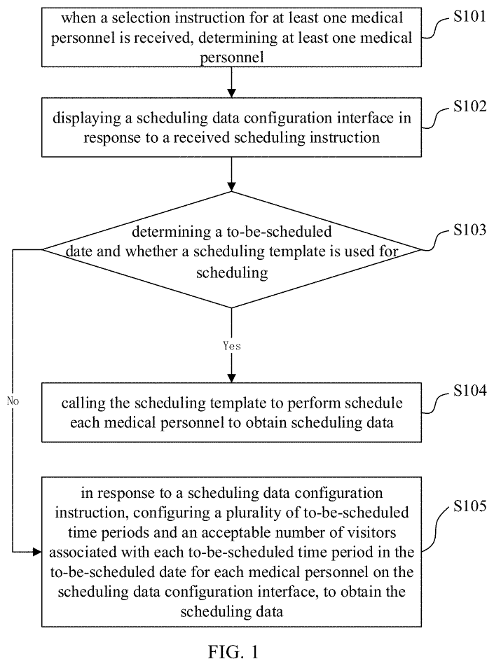

The embodiment of the present disclosure provides a medical personnel information management method, which can be applied to a medical personnel management system (such as an internet hospital system), and as shown in FIG. 1, the method includes:

S101, when a selection instruction for at least one medical personnel is received, determining at least one medical personnel.

The selection instruction may be, for example, a selection operation performed by the user on a list of displayed medical personnel or a list of departments. The user may select one or more medical personnel to be scheduled on the displayed list of medical personnel or one or more departments to be scheduled on the displayed list of departments. In an alternative embodiment, when a user selects a department from the list of departments, all medical personnel in that department may be selected by default; in another alternative embodiment, when the user selects a department in the list of departments, a list of all medical personnel for that department may be presented, and the user may select one or more medical personnel to be scheduled in the presented list of department departments.

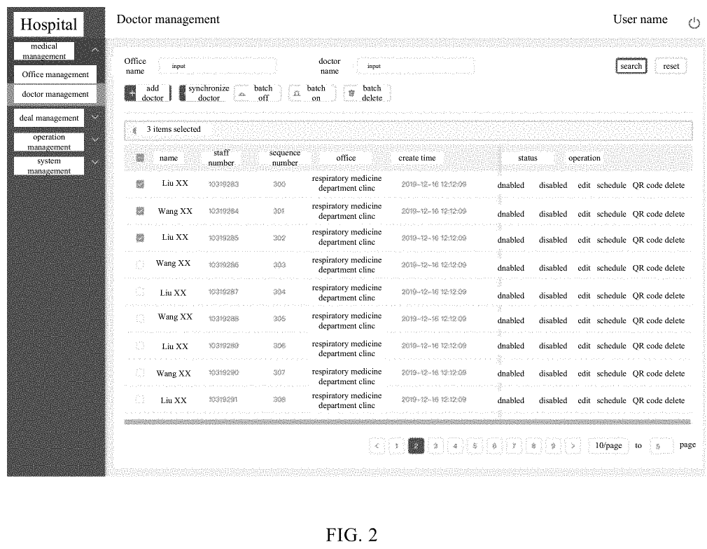

The medical personnel list can be shown in the form shown in FIG. 2, in which the name, job number, sequence number, department, creation time, and status (for example only, other information may also be shown) of each medical personnel are shown, the left side of the information of each medical personnel is shown with a checkbox, the right side of the information of each medical personnel is shown with four operation options (or icons) of “disable (or enable)”, “edit”, “shift”, “two-dimensional code”, and “delete”, the upper side of FIG. 2 is shown with operation options (or icons) of “add doctor”, “synchronize doctor”, “batch disable”, “batch enable”, “batch delete”, and the like, and input boxes of “department name” and “doctor name”, and the function of each operation option (icon) and input box is as follows:

Inputting corresponding department names in an input box of the department names, triggering (for example, clicking or touching) a query option, enabling a lower medical personnel list to display information of all medical personnel of the department, and displaying the information of all medical personnel of all departments in a default manner when no input is made; inputting the name of a medical care worker in the input box of the doctor name, triggering the query option and enabling the medical care worker list below to display the information of the medical care worker; triggering the “reset” option may reset the contents in both input boxes; triggering the check box of one or more medical personnel in the medical personnel list, selecting the medical personnel, and triggering the check box again can cancel the selected operation.

For a certain medical personnel, the state of the medical personnel can be changed into a non-use state from a use state by triggering a non-use option, the medical personnel can be scheduled in the use state, and the medical personnel cannot be scheduled in the non-use state; the information of the medical personnel can be modified by triggering the ‘editing’ option; the configuration of the scheduling data can be carried out on a scheduling configuration interface of the medical personnel by triggering the ‘scheduling’ option; triggering the two-dimension code option can display the two-dimension code information of the medical personnel; triggering the “delete” option may delete the medical personnel's information.

For the selected three medical personnels as shown in FIG. 2, the activation state of the three medical personnels can be changed into the deactivation state by triggering the batch deactivation, and the deactivation state of the three medical personnels can be changed into the activation state by triggering the batch activation; the information of three medical personnel can be deleted at the same time by triggering batch deletion; triggering the “add doctor” option may add new medical personnel information to the system; triggering the ‘synchronous doctor’ option can integrally import medical personnel information in other systems into the system.

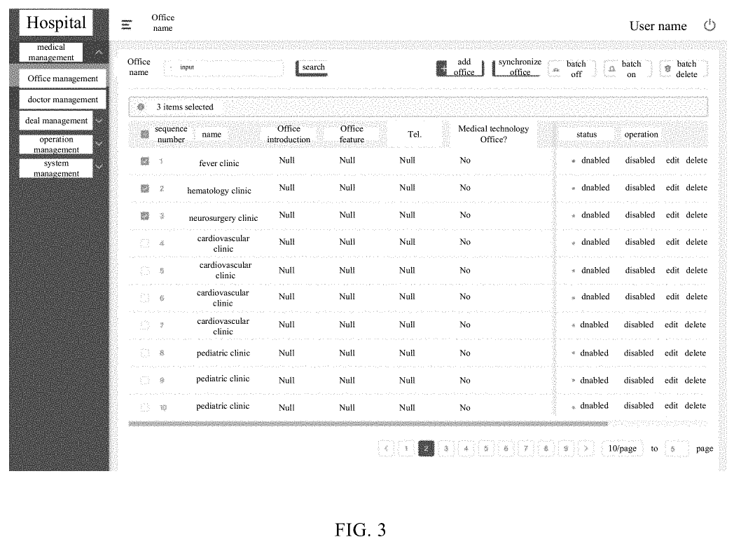

The department list can be shown as shown in FIG. 3, in the list, the serial number, name, department introduction, department feature, contact telephone, medical department, status (for example only, other information may also be shown) of each department are shown, the left side of the information of each department shows a checkbox, the right side shows three operation options of “stop (or start)”, “edit”, and “delete”, the upper part of FIG. 3 shows operation options of “add department”, “synchronize department”, “batch stop”, “batch start”, “batch delete”, and the like, and input boxes of “department name”, and the functions of the operation options and input boxes are as follows:

inputting corresponding department name in the input box of the department name, triggering (for example, clicking or touching) a search option on the right side of the input box, so that the information of the department can be displayed on a department list below the input box, and the information of all departments can be displayed by default when no input is made; and triggering check boxes of one or more departments in the department list, selecting the department, and triggering the check boxes again to cancel the selected operation.

For a department, triggering a ‘non-use’ option can change the state of the department from an activation state to a non-use state, the medical personnel of the department can be scheduled in the activation state, and the medical personnel of the department cannot be scheduled in the non-use state; the information of the department can be modified by triggering the ‘editing’ option; triggering the “delete” option may delete the information for that department.

For the selected three departments as shown in FIG. 3, triggering “batch deactivation” may change the activation state of the three departments to the deactivation state at the same time (no modification is needed if the deactivation state exists in the selected department), and triggering “batch activation” may change the deactivation state of the three departments to the activation state at the same time (no modification is needed if the activation state exists in the selected department); triggering batch deletion can delete the information of three departments at the same time; the information of a new department can be added into the system by triggering the option of ‘newly adding departments’; triggering the option of ‘synchronous department’ can lead the information of departments in other systems into the system in whole.

The information shown in FIG. 2 and FIG. 3 is only an example, and the corresponding information may be modified or added according to actual requirements. For example, an option of “integral scheduling” may be added to the interface shown in FIG. 2, and when the option of “integral scheduling” is triggered, the entry into the scheduling data configuration interface is performed to perform integral scheduling on the selected multiple medical personnel (for example, in the case where multiple medical personnel use the same set of scheduling data); for another example, a “reset” option may be added to the interface shown in FIG. 3, a “scheduling” option may be added after information of each department, and when the “scheduling” option is triggered, the interface enters a scheduling data configuration interface to perform scheduling on medical personnel of a corresponding department.

S102, displaying a scheduling data configuration interface in response to a received scheduling instruction.

The shift instruction in the embodiment of the present disclosure may be a trigger operation on the “scheduling” option shown in FIG. 2, and if the “overall scheduling” option is added in FIG. 2 and the “scheduling” option is added in FIG. 3, the scheduling instruction may also be a trigger operation on any one of the added options.

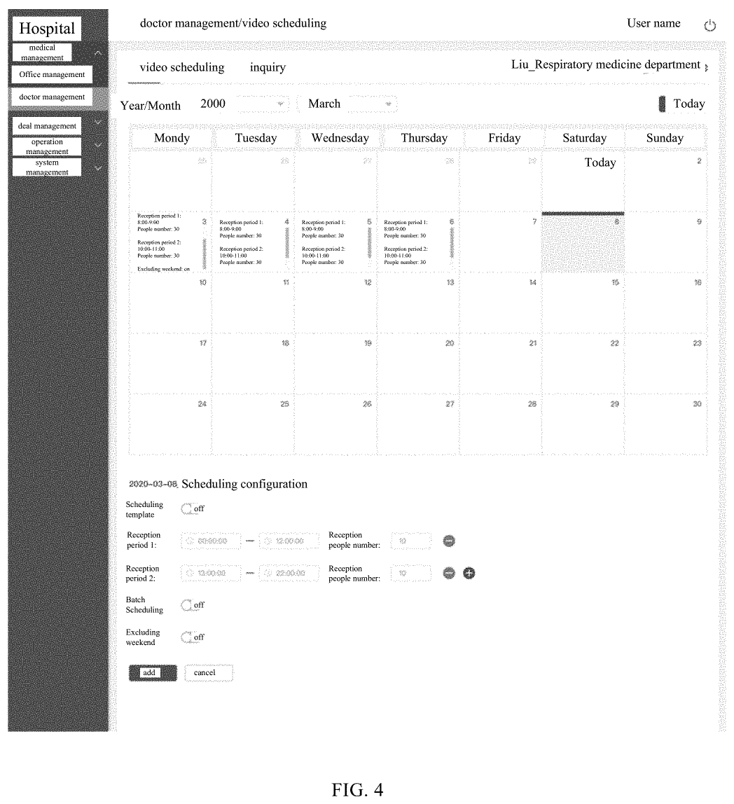

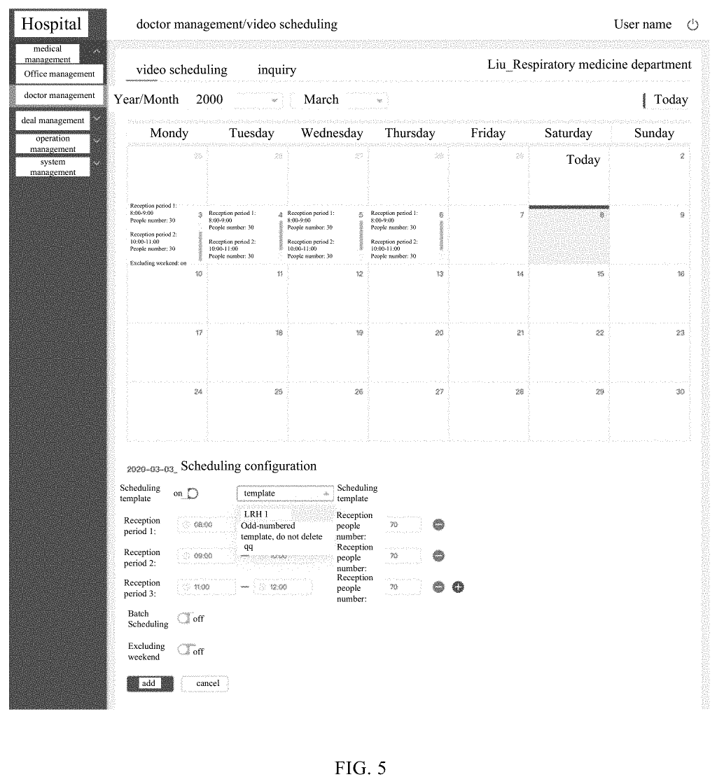

The configuration interface of scheduling data for scheduling a medical personnel can be shown in a form shown in FIG. 4 or FIG. 5, a calendar is shown above the interface and can be used for selecting specific dates to be scheduled, the specific form (month or year) shown by the calendar can be set according to actual requirements, and specific configuration options of the scheduling data, such as ‘scheduling template’, ‘batch scheduling’, ‘queuing weekend’, etc., are shown below the interface and will be introduced in combination with specific application scenes.

S103, determining a to-be-scheduled date and whether a scheduling template is used for scheduling; if yes, go to step S104, otherwise go to step S105.

The shift instruction in the embodiment of the present disclosure may be a trigger operation on the “scheduling” option shown in FIG. 2, and if the “overall scheduling” option is added in FIG. 2 and the “scheduling” option is added in FIG. 3, the scheduling instruction may also be a trigger operation on any one of the added options.

The configuration interface of scheduling data for scheduling a medical personnel can be shown in a form shown in FIG. 4 or FIG. 5, a calendar is shown above the interface and can be used for selecting specific dates to be scheduled, the specific form (month or year) shown by the calendar can be set according to actual requirements, and specific configuration options of the scheduling data, such as ‘scheduling template’, ‘batch scheduling’, ‘queuing weekend’, etc., are shown below the interface and will be introduced in combination with specific application scenes.

Optionally, determining the to-be-scheduled date includes: and determining the selected date as the to-be-scheduled date or taking the current date as the to-be-scheduled date in response to the selection instruction for the date.

Referring to the examples shown in FIGS. 4 and 5, the selection instruction may be a user selection operation on a certain date on the displayed calendar, and when the selection instruction is received, the selected date is taken as the to-be-scheduled date; when no selection instruction is received, the current date can be used as the to-be-scheduled date by default. The specific date information of the current date can be determined according to the local time acquired by the front end, and the to-be-scheduled date in FIGS. 4 and 5 is the current date 2020, 3, 8.

Alternatively, when determining whether to perform scheduling using the scheduling template, the determination may be made based on a setting operation (which may be implemented by any one of clicking, touching, sliding, and the like) of a user on a switch option of the “scheduling template” shown in FIGS. 4 and 5, if the switch option of the “scheduling template” is set to on (as shown in FIG. 5), it may be determined to perform scheduling using the scheduling template, and if the switch option of the “scheduling template” is set to off (as shown in FIG. 4), it may be determined not to perform scheduling using the scheduling template.

S104, when the scheduling template is determined to be used for scheduling, calling the scheduling template to perform schedule each medical personnel to obtain scheduling data.

The scheduling template in the embodiment of the application includes to-be-scheduled time periods in a day to be scheduled and the acceptable number of visitors associated with each to-be-scheduled time period, and in some embodiments, the scheduling template can also include a template name. The scheduling templates can be configured and stored in advance, the number of the stored scheduling templates is at least one, and a plurality of scheduling templates can be distinguished from one another based on the names of the templates.

Optionally, when a shift template is required to be used for shifting, a required shift template can be selected from the displayed template selection frame, and an interface is called to obtain data of the shift template, so that the shift efficiency is improved. The scheduling data obtained by using the scheduling template can be modified according to actual requirements, such as adding, modifying or deleting some part of data.

Referring to the scheduling data configuration interface shown in FIG. 5, when the switch option of the “scheduling template” is set to on, the scheduling data configuration interface may synchronously display the template query frame and the “configuration template” option, the stored scheduling template may be queried and called through the template query frame, and a new scheduling template may be configured through the “configuration template” option.

When the scheduling template is inquired and called, the template name of the scheduling template to be inquired and called is input in the template inquiry frame, the template name is correspondingly displayed in a pull-down menu, and the scheduling data of the scheduling template corin response to the template name can be called after the template name is selected; or, the template query box can be selected, the stored template names of all the scheduling templates can be correspondingly displayed in the pull-down menu, and the scheduling data of the scheduling template corin response to the template name can be called after the required template name is selected.

Optionally, the scheduling template in the embodiment of the present disclosure is configured in the following manner: in response to a template configuration instruction, and configuring at least one template name in a template configuration interface; and for each template name, configuring and storing a plurality of to-be-scheduled time periods and the number of available doctors associated with each to-be-scheduled time period. The template configuration instruction can be a trigger operation of a user on a configuration template option in the scheduling data configuration interface.

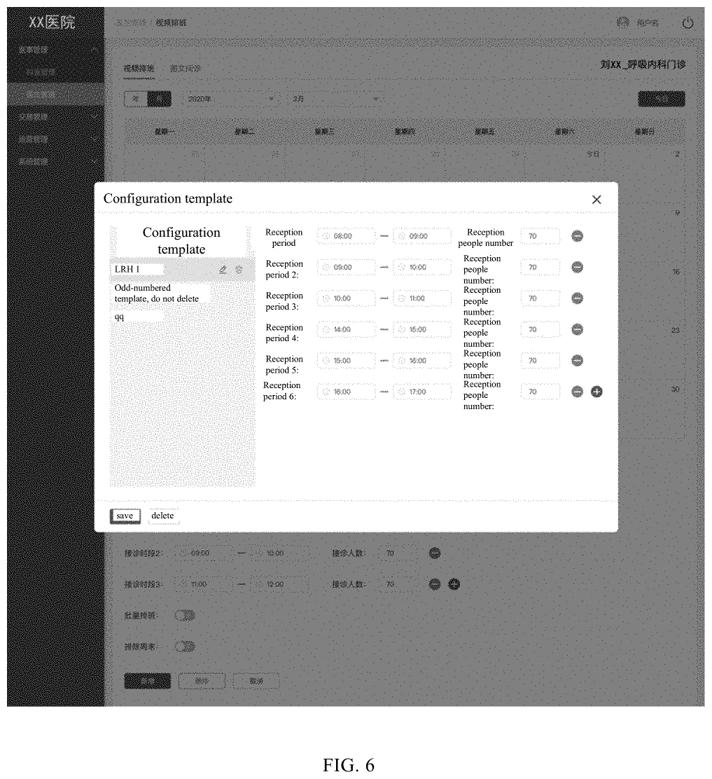

In a specific example, in a case that no shift template is pre-stored or a stored shift template cannot meet a requirement, a new shift template may be configured and stored by triggering a “configuration template” option in the interface shown in FIG. 5, which is specifically as follows: after the option of configuring the template is triggered, jumping to a template configuration interface shown in FIG. 6, configuring the template name of each shift template on the template configuration interface by triggering the option of +adding the template, then configuring the starting time and the ending time of each time period to be shifted and the number of available doctors in the time period to be shifted for each template name, and calling the interface to be stored to the server by triggering the option of storing.

The configured data can be modified or deleted according to the actual demand, and the configured scheduling templates can be called in the mode according to the newly increased data of the actual demand.

In the template configuration interface shown in FIG. 6, the current template configuration may also be cancelled by triggering a “cancel” option.

S105, when the scheduling template is determined not to be used for scheduling, in response to a scheduling data configuration instruction, configuring a plurality of to-be-scheduled time periods and an acceptable number of visitors associated with each to-be-scheduled time period in the to-be-scheduled date for each medical personnel on the scheduling data configuration interface, to obtain the scheduling data.

Optionally, in a case that it is determined that a shift template is not used for performing a shift, after configuring a plurality of to-be-scheduled time periods in dates to be scheduled and the acceptable number of visitors associated with the to-be-scheduled time periods in response to a scheduling data configuration instruction, the to-be-scheduled time periods may be stored as a new shift template, and this operation may be implemented as follows: on the configuration interface of the scheduling data shown in FIG. 4, an option of “save as template” is added, and the user can complete the operation when triggering the option.

Referring to the example shown in FIG. 4, the scheduling data configuration instruction may be an input operation to an input box of the attendance period 1 (as one period to be scheduled), the attendance period 2 (as another period to be scheduled), the number of people in attendance (as the number of people that can be attended), and the like in FIG. 4.

Optionally, the medical personnel information management method provided in the embodiment of the present disclosure further includes:

determining whether to perform batch scheduling; when the batch scheduling is determined, a plurality of target dates which take the to-be-scheduled dates as the starting points and are within the preset batch scheduling date range are determined, and the scheduling data are applied to each target date.

For example, in some embodiments, when determining to perform a batch shift, a plurality of target dates may be determined by selecting a date as a starting point of the shift date and another date as an ending point of the shift date, i.e., a plurality of dates between the starting point and the ending point date as target dates, and applying scheduling data to each target date.

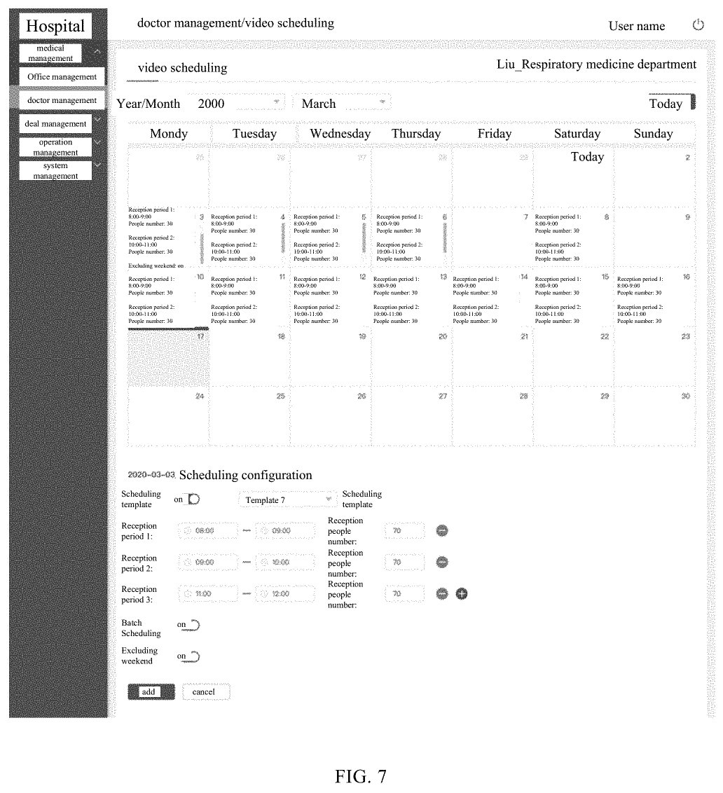

Referring to the interfaces shown in FIGS. 4, 5, and 7, whether or not to perform the batch shift may be determined based on a setting operation (which may be performed by any one of clicking, touching, sliding, and the like) of the switch option of the “batch shift” by the user, and if the switch option of the “batch shift” is set to on (as shown in FIG. 7), the batch shift may be determined, and if the switch option of the “batch shift” is set to off (as shown in the figure or as shown in FIG. 7), the batch shift may not be determined.

Optionally, determining a plurality of target dates which are within the batch shift date range and start from the to-be-shifted date includes: and acquiring at least one scheduling date strategy, and determining a plurality of dates which accord with a preset batch scheduling date range and each scheduling date strategy by taking the to-be-scheduled date as a starting point to be used as a plurality of target dates. For example, in embodiments where multiple dates between the selected start and end dates are determined as target dates, shift date policies may also be incorporated.

The at least one scheduling date strategy includes at least one of: date excepting weekends, date excepting holidays, each date in a specified range of consecutive dates reserved, odd dates reserved, even dates reserved.

The holidays in the embodiment of the present disclosure refer to statutory holidays of the country.

In the embodiment of the present disclosure, the specified continuous date range may be a specified date range of a certain month or a certain week. If the specified month is the current month, the specified continuous date range may be a date range after the current date in the current month; if the specified week is the current week, the specified continuous date range may be a date range after the current date in the current week. Referring to the calendar shown in FIG. 7, if the designated month is 2020 year 3 month (i.e., the current month) shown in FIG. 7, the designated consecutive date range may include 3 month 17 days to 3 month 30 days (including two end point dates) as shown in FIG. 7; if the specified week is the fourth week of 2020 month 3 (assuming that Monday is the calculation start point of one week and Sunday is the calculation end point of one week), as shown in FIG. 7, the specified continuous date range includes days 3 month 17 to 3 month 23 (including two end dates) as shown in FIG. 7.

In the embodiment of the present disclosure, the specified continuous date range may also be a continuous date range starting from the current date, for example, a month starting from the current date (shift by month) or a week starting from the current date (shift by week). Referring to the calendar shown in FIG. 7 (2020, 3 months), if the shift is performed monthly, the specified continuous date range includes 3 months 17 to 4 months 16 (including two end dates), and if the shift is performed weekly, the specified continuous date range includes 3 months 17 to 3 months 23 (including two end dates).

The scheduling date strategies can be preset and stored, the required scheduling date strategy is obtained through a setting operation calling interface of each scheduling date strategy option when needed, and the scheduling to-be-scheduled date is determined based on the scheduling to-be-scheduled date, a preset batch scheduling date range and the scheduling date strategy; when the obtained scheduling date strategies are more than two, an intersection can be taken from the date ranges of the two scheduling date strategies, and then the target date of batch scheduling is determined by combining the to-be-scheduled date and the preset batch scheduling date range.

In one example, when the obtained two scheduling date policies are dates excluding weekends and scheduled monthly with the current date as a starting point, firstly, an intersection is taken from date ranges corin response to the two scheduling date policies to obtain a date range except for one month with the current date as the starting point, and then, each date after the selected date in the date range is used as a target date for batch scheduling, so that batch scheduling is performed on the target date to improve scheduling efficiency.

Referring to the calendar (3 months in 2020) shown in FIG. 7, when the two acquired shift schedule policies are dates excluding weekends and shift monthly from the current date, intersection is taken for the date ranges corin response to the two shift schedule policies, and the obtained date ranges include all dates from Monday to Friday between 17 days in 3 months and 16 days in 4 months (including two end-point dates), and include, within the range of 3 months, 17 days in 3 months to 21 days in 3 months and 24 days in 3 months to 28 days in 3 months.

For example, in embodiments where multiple dates between the selected start and end dates are determined as target dates, shift date policies may also be incorporated. For example, after at least one of the date excluding weekends, the date excluding holidays, each date within a continuous date range specified by retention, the odd-numbered date remaining, the even-numbered date remaining, and the like is performed on a plurality of dates between the starting point and the ending point as the target date.

In the examples shown in FIGS. 4, 5 and 7, only one shift date strategy option, namely, the “weekend excluded” switch option is shown, and when both the “batch shift” and “weekend excluded” switch options are set to on (as shown in FIG. 7), the corresponding interface may be invoked to obtain the strategy of queuing weekends, otherwise the corresponding interface is not invoked to obtain the strategy of queuing weekends. In other examples, other shift date strategy options may be added in any of the examples of FIGS. 4, 5, and 7, such as adding switch options such as “exclude holidays”, “shift by month”, “shift by week”, and so on.

The batch scheduling date range in the embodiment of the application can be preset according to actual requirements and is associated with the switch option of batch scheduling, and when the switch option of batch scheduling is set to be on, the preset batch scheduling date range can be called.

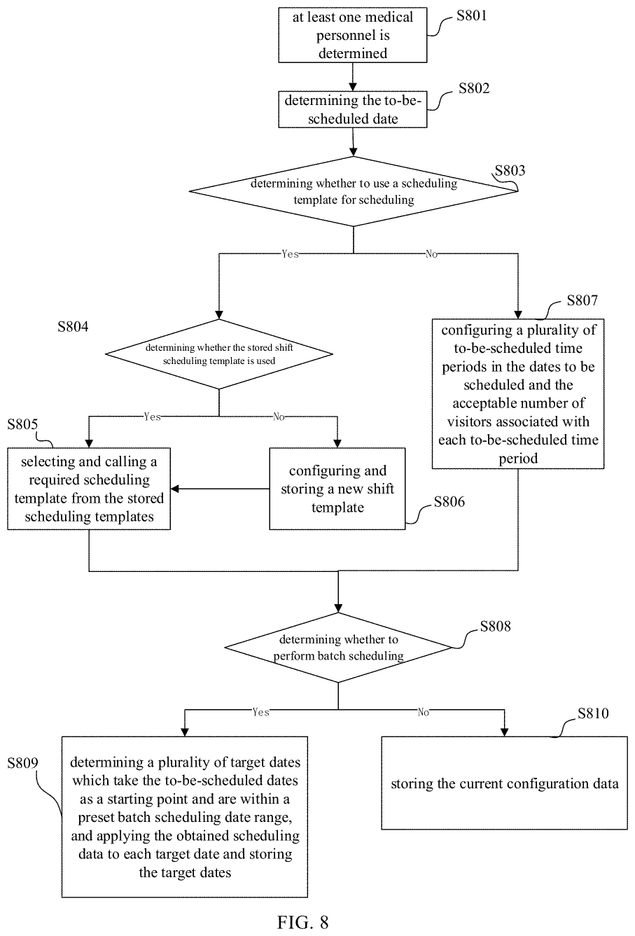

FIG. 8 shows an expansion flow of an alternative implementation of the medical personnel information management method provided by the embodiment of the present disclosure, which includes the following steps S801-S810:

s801, at least one medical personnel is determined.

S802, determining the to-be-scheduled date.

S803, determining whether to use a scheduling template for scheduling; if yes, go to step S804, otherwise go to step S807.

S804, determining whether the stored scheduling template is used; if yes, go to S805, otherwise go to S806.

S805, selecting and calling a required scheduling template from the stored scheduling templates.

S806, configuring and storing a new shift template, and then executing S805.

S807, configuring a plurality of to-be-scheduled time periods in the dates to be scheduled and the acceptable number of visitors associated with each to-be-scheduled time period.

And configuring a starting time and an ending time in each period of time to be scheduled.

S808, determining whether to perform batch scheduling; if yes, go to S809, otherwise go to S810.

S809, determining a plurality of target dates which take the to-be-scheduled dates as a starting point and are within a preset batch scheduling date range, and applying the obtained scheduling data to each target date and storing the target dates.

Referring to the example shown in FIG. 7, the operation of applying the obtained scheduling data to each target date in the embodiment of the present disclosure may be implemented by triggering an “add” icon on the interface shown in FIG. 8.

S810, storing the current configuration data.

Referring to the example shown in FIG. 4, saving the current configuration data may be accomplished by triggering an “Add New” icon on the interface shown in FIG. 4.

The details of the steps shown in FIG. 8 can be found in the detailed description above.

In the embodiment of the present disclosure, the triggering operation on each operation option or icon may be implemented in any one of a click, a touch, a slide, and the like, depending on a specific presentation manner, which is not limited in the embodiment of the present disclosure.

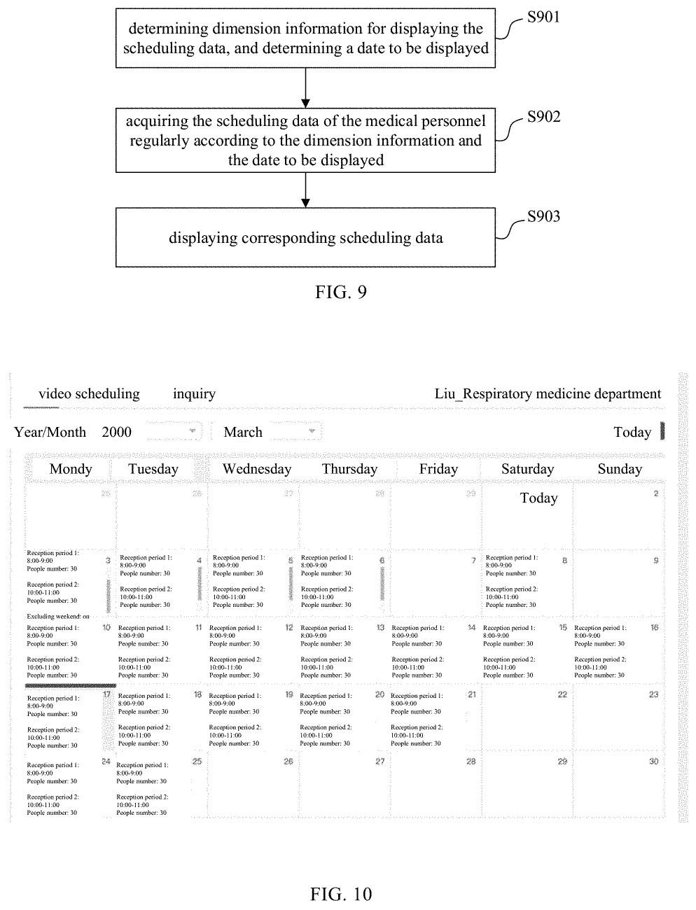

Optionally, as shown in FIG. 9, the medical personnel information management method provided in the embodiment of the present disclosure further includes, for example, the following steps S901 to S903:

S901, determining dimension information for displaying the scheduling data, and determining a date to be displayed.

The dimension information includes at least one of the name of the medical personnel and the department to which the medical personnel belongs, when the related data is displayed subsequently, when the dimension information only includes the name of the medical personnel, the data of the medical personnel is only displayed, when the dimension information only includes the department to which the medical personnel belongs, the data of the department is only displayed, and when the dimension information simultaneously includes the name of the medical personnel and the department to which the medical personnel belongs, the data of the medical personnel and the department to which the medical personnel belongs can be displayed simultaneously.

The date to be shown can be determined according to actual requirements, can be the current date, can also be other dates before or after the current date (in the case that scheduling data exists on the selected date), can be one date, and can also include a plurality of dates.

S902, acquiring the scheduling data of the medical personnel regularly according to the dimension information and the date to be displayed.

The time interval for acquiring the scheduling data can be set according to actual requirements, and in one example, the interface is called every 5 minutes to acquire the latest scheduling data so as to update the data.

S903, displaying corresponding scheduling data.

When the dimension information only includes the name of the medical personnel, the scheduling data of the medical personnel can be only displayed, when the dimension information only includes the department to which the medical personnel belongs, the scheduling data of the department can be only displayed, and when the dimension information simultaneously includes the name of the medical personnel and the department to which the medical personnel belongs, the scheduling data of the medical personnel and the department to which the medical personnel belongs can be simultaneously displayed.

In one example, if the date to be displayed is the current date, the scheduling data of the medical personnel or the department on the current date is displayed, and if the date to be displayed includes all the dates of the current week, the scheduling data of all the dates of the medical personnel or the department in the current week is displayed.

Optionally, the scheduling data can be displayed in the form of a calendar, a chart and the like, so that the scheduling data is convenient to view. FIG. 10 shows a schematic diagram of displaying scheduling data of a doctor in the form of a calendar, in which each visit period (i.e., scheduling period) and the number of available visits in each visit period are displayed in each date to be displayed in the calendar. The display mode shown in FIG. 10 may also be applied to a scheduling stage, for example, the scheduling data configuration interface shown in FIG. 4, FIG. 5, and FIG. 7, the scheduling data of each configured date before the to-be-scheduled date may be displayed in the scheduling data configuration interface, and the previous scheduling data may be viewed in real time as the data reference of this scheduling during scheduling, which is beneficial to optimizing scheduling.

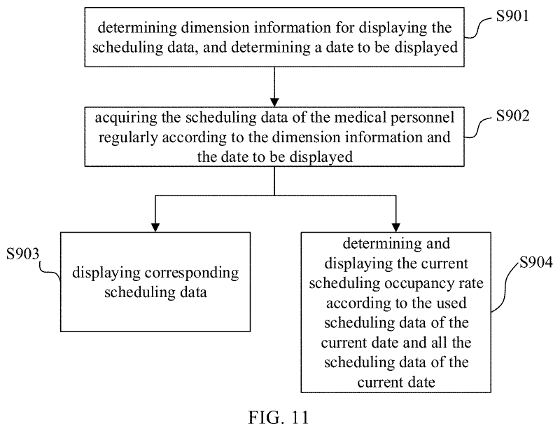

Optionally, as shown in FIG. 11, the medical personnel information management method provided in the embodiment of the present disclosure further includes, for example, the following step S904:

S904, determining and displaying the current scheduling occupancy rate according to the used scheduling data of the current date and all the scheduling data of the current date.

The used scheduling data is data which is actually used before the current time in the scheduling data of the current date.

In an optional embodiment, the used shift schedule data of the current date includes the number of people who have actually received before the current time in the current date (referred to as the number of people who have been visited for short, and may be referred to as the number of people who have been visited in a hospital application scenario), all shift schedule data of the current date includes the number of people who can be visited in the shift schedule data of the current date, and the current shift schedule occupancy rate may be determined according to the following manner:

in this way, the personal shift occupancy of a selected medical personnel and/or the overall shift occupancy of a department may be determined and displayed. When the personal duty ratio of a certain medical personnel is determined, the personal duty ratio of the medical personnel can be determined according to the number of visitors who have received a visit on the current date of the medical personnel and the number of visitors who can receive a visit on the current date of the medical personnel; when the integral duty-cycle rate of a certain department is determined, the integral duty-cycle rate of the department can be determined according to the number of the patients who have received a visit on the current date of all medical personnels in the department and the number of the patients who can receive a visit on the current date of all medical personnels in the department.

The scheduling utilization rate can be displayed in the scheduling data displayed in the forms of a calendar, a chart and the like in the forms of a progress bar, a pie chart and the like.

The determination and the display of the scheduling occupancy rate are beneficial to timely knowing the service condition of the scheduling data, and reference data is provided for subsequent work and scheduling.

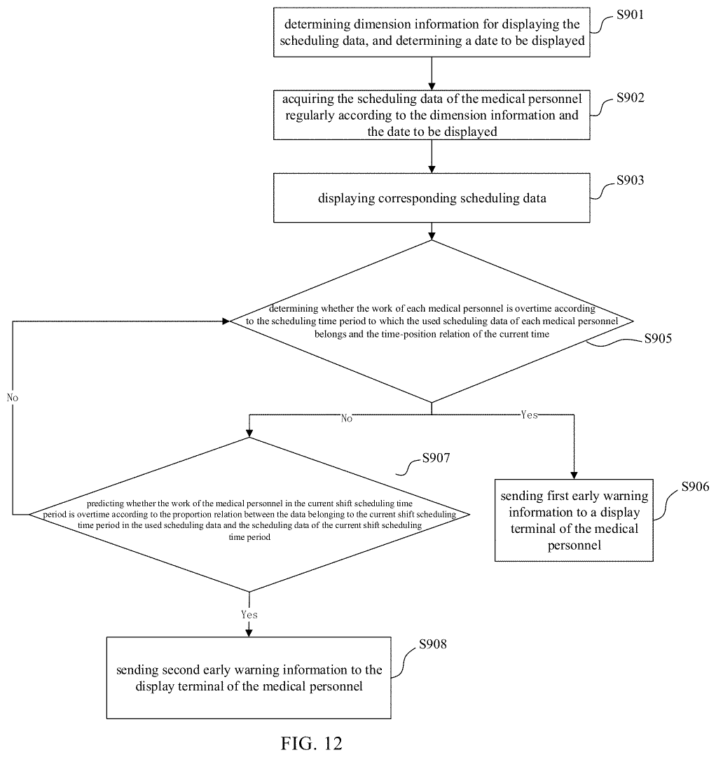

Optionally, as shown in FIG. 12, the medical personnel information management method provided in the embodiment of the present disclosure further includes, for example, the following steps S905 to S908:

S905, determining whether the work of each medical personnel is overtime according to the scheduling time period to which the used scheduling data of each medical personnel belongs and the time-position relation of the current time; if yes, go to step S906; if not, S907 is executed.

The scheduling time period in the embodiment of the application is the same as the time range of the to-be-scheduled time period in the scheduling stage, and after the scheduling is finished, the to-be-scheduled time period can be called the scheduling time period.

Optionally, for each medical personnel, if the scheduling time period using the scheduling data does not include the current time, it may be determined that the scheduling time period of the medical personnel has been overtime, and if the scheduling time period using the scheduling data includes the current time, it may be determined that the scheduling time period of the medical personnel has not been overtime.

Taking a hospital scene as an example, assuming that the scheduling time period to which the used scheduling data of the doctor A belongs is 8:00-10:00 and the current time is 10:15, the scheduling time period of the doctor A can be determined to be overtime, and under one condition, if the 10:00-10:15 is preset rest time without scheduling, the scheduling time period is not determined to be overtime; assuming that the used scheduling data of doctor A has a scheduling time period of 8:00-10:00 and the current time is 9:30, it is determined that the scheduling time period of doctor A has not timed out.

And S906, sending first early warning information to a display terminal of the medical personnel.

The first early warning information can be displayed by the display terminal to remind medical personnel that the work is overtime and needs to be processed as soon as possible; the first early warning information can be displayed in any form of characters, symbols, figures, colors and the like.

S907, predicting whether the work of the medical personnel in the current scheduling time period is overtime according to the proportion relation between the data belonging to the current scheduling time period in the used scheduling data and the scheduling data of the current scheduling time period; if yes, go to S908; if not, go to S905.

Taking a hospital scene as an example, assume that the current scheduling time period is 9:00-9:30, and the current time is 9:15, the number of available doctors of the doctor A is 10 in the current scheduling time period of 9:00-9:30, and the number of the available doctors is 10 at the current time 9:15, the number of people who belong to the current shift time period 9:00-9:30 is 4, the ratio of the number of people who belong to the current shift time period 9:00-9:30 to the number of people who can be diagnosed in the current shift time period 9:00-9:30 is 40%, and for the current shift time period, when the time is half, the number of people who have been diagnosed is not half, and the change of the time is slower, the diagnosis receiving work of the doctor A in the current time period can be predicted to be overtime.

S908, sending second early warning information to the display terminal of the medical personnel.

The second early warning information can be displayed by the display terminal to remind medical personnel that the current work may be overtime and needs to be processed as soon as possible; the second early warning information can be displayed in any form of characters, symbols, figures, colors and the like.

The second warning information may be displayed in a different form from the first warning information, or in a different degree in the same form, for example, a graphic with a different shape or size, a different color, and a color with a different depth, so that the user can distinguish between the overtime condition and the possible overtime condition and take different countermeasures.

Optionally, the medical personnel information management method provided in the embodiment of the present disclosure further includes: and in response to the information query instruction for the current scheduling time period, and displaying at least one of reservation information, used scheduling data and to-be-used scheduling data of the current scheduling time period.

The to-be-used scheduling data is data except used scheduling data in all the scheduling data of the current date.

The information query instruction in the embodiment of the present disclosure may be generated based on an information query operation of a user, where the information query operation of the user may be at least one of the following operations: and clicking or touching a progress bar of the scheduling utilization rate, clicking or touching scheduling data of the current scheduling time period, clicking or touching scheduling data of any one scheduling time period, and the like, which can be specifically set according to actual requirements.

In one example, upon receiving an information query instruction for a current scheduling time period, at least one of registration information (as reservation information), a number of people who have received a visit (as used scheduling data), and queuing information (as to-be-used scheduling data) of the current scheduling time may be presented.

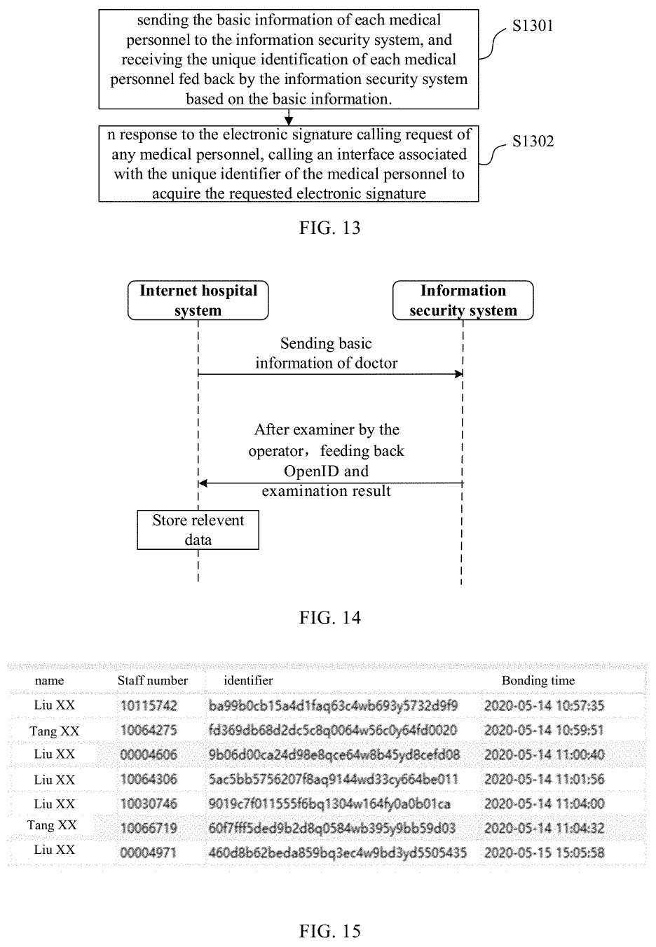

Optionally, as shown in FIG. 13, the medical personnel information management method provided in the embodiment of the present disclosure further includes steps S1301-S1302;

S1301, sending the basic information of each medical personnel to the information security system, and receiving the unique identification of each medical personnel fed back by the information security system based on the basic information.

The basic information of the medical personnel includes at least one of information of the name, the job number, the department, the title and the contact information of the medical personnel.

The information security system in the embodiment of the present disclosure may be a CA (security Authority) system, and the unique identifier may be an OpenID (a digital identity framework with a user as a center).

Optionally, after receiving the unique identifier of each medical personnel fed back by the information security system, the medical personnel's login account number (which may be a name, a job number, or an account number in other forms) and the unique identifier may be bound, and the bound login account number and unique identifier are returned to the information security system and stored in the database after being checked by the information security system. After the medical personnel logs in the Internet system through the login account, the unique identification of the medical personnel can be found according to the login account of the medical personnel, so that information can be called conveniently through the unique identification, and safety is improved.

FIG. 14 shows an information interaction method between an internet hospital system and a CA system, where the internet hospital system sends basic information of medical care personnel to the CA system to implement information synchronization, the CA system feeds back a unique identifier OpenID and an audit result to the internet hospital system after receiving the information and auditing by an operator, and the internet hospital system stores relevant data.

FIG. 15 shows a schematic diagram of the job number (docctorcode) and unique identification OpenID binding of a medical personnel, in the example of FIG. 15, the job number (docctorname) and unique identification OpenID of 7 medical personnels are bound, and FIG. 15 also shows the name (docctorname) and binding time (createTime) of the 7 medical personnels.

S1302, in response to the electronic signature calling request of any medical personnel, calling an interface associated with the unique identifier of the medical personnel to acquire the requested electronic signature.

Optionally, when an electronic signature calling request of any medical personnel is received, sending the electronic signature calling request to the information security system; in response to an agreeing calling instruction fed back by the information security system, and calling an interface associated with the unique identifier of the medical personnel to acquire the electronic signature of the medical personnel; in response to a call rejection instruction fed back by the information security system, displaying an authorization interface for judging whether to authorize to call the electronic signature, in response to an authorization approval instruction, calling an interface associated with the unique identifier of the medical personnel to acquire the electronic signature of the medical personnel, and uploading authorization information to the information security system.

The consent calling instruction is generated by the information security system when the electronic signature is verified to be authorized, and the denial calling instruction is generated by the information security system when the electronic signature is verified to be unauthorized.

In the embodiment of the application, the communication token can be called based on the obtained interface to call the interface associated with the unique identifier of any medical personnel so as to obtain the electronic signature of the medical personnel, and the interface calling communication token can be dynamically generated and obtained through a dynamic authorization token generation mechanism so as to reduce the pressure of interface access of the information security system and improve the efficiency. The dynamic authorization token generation mechanism in the embodiment of the present disclosure may generate an interface call communication token periodically and store the interface call communication token in the database, for example, a new interface call communication token that is automatically generated every 1 hour is stored in the database.

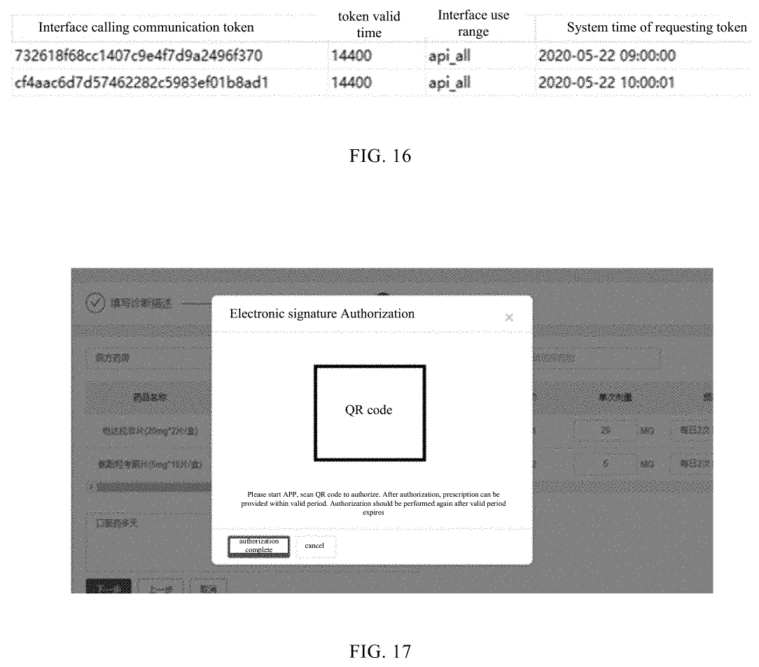

FIG. 16 shows the two interface calls generated dynamically to communicate the token (accessToken), the valid time (in seconds) of the token (expires), the scope of use of the information security system configuration interface (scope), and the system time (creatTime) at which its internet hospital system requests authorization to the token.

FIG. 17 is a schematic diagram of an authorization interface for determining whether to authorize the electronic signature, where a user can scan a two-dimensional code (information of the two-dimensional code is not directly shown) in the two-dimensional code area in FIG. 17 to authorize the electronic signature, and the information of the two-dimensional code can be provided by an information security system.

In the embodiment of the application, the electronic signature is authorized to indicate that the electronic signature is authorized by the person and is within the authorized validity period, and the electronic signature is not authorized by the person or authorized by the person and is not within the validity period.

The electronic signature can be agreed to be called only when the authorization of a certain electronic signature is verified, so that the use safety of the electronic signature is ensured, and the electronic signature is prevented from being stolen; when the electronic signature is verified to be unauthorized, the opportunity of re-authorization is provided so as to ensure the normal use of the user, and the convenience can be improved while the safety is ensured.

In one example, doctor a needs to use an electronic signature when making a prescription (or medical orders and other electronic documents needing to use the electronic signature) at the doctor end of the internet hospital, the internet hospital system sends the electronic signature calling request to the information security system when receiving the electronic signature calling request of the doctor, and the information security system firstly verifies whether doctor a starts automatic signature authorization and whether the authorization is in a valid period; if the doctor is authorized and within the validity period, an agreement calling instruction is fed back to the internet hospital system, the internet hospital system calls an interface associated with the unique identification OpenID of the doctor A to obtain the electronic signature of the doctor A, the electronic signature is added to the doctor signature part of the prescription, and the signed prescription shown in the graph 18 is formed at the doctor end; if the two-dimension code is not authorized or the authorized validity period is over, the two-dimension code automatic signature authorization page shown in FIG. 17 is fed back to the doctor A through the internet hospital system, and the doctor A can scan the two-dimension code for authorization or re-authorization.

In an optional embodiment, when a doctor A orders for the first time, code scanning authentication and authorization are required to be performed through any one of doctor terminals of the information security system in the above mode; the step of scanning code authentication authorization may be skipped for a secure period (which may be set in advance in the information security system, e.g., 30 minutes) other than when the first order is placed.

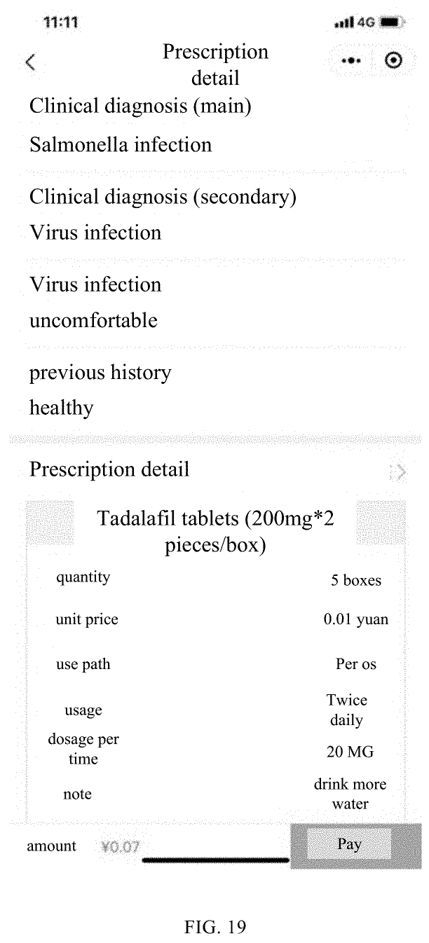

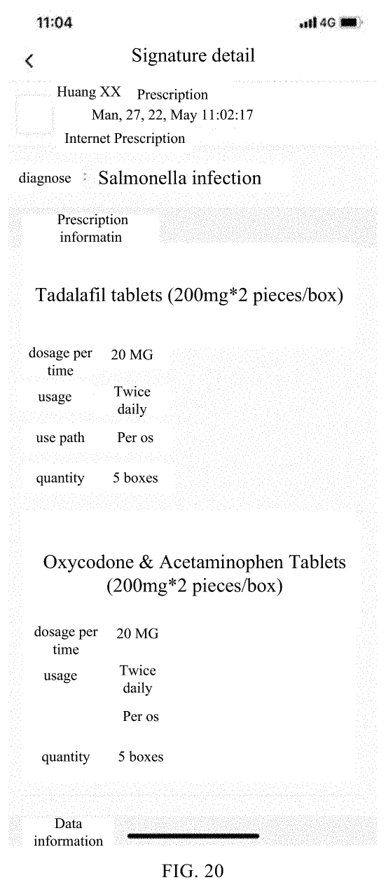

The signed prescription information of the doctor end can be synchronized to the patient end of the internet hospital system for patient inquiry, and the prescription interface of the patient inquiry is shown in FIG. 19; the signed prescription can also be synchronously uploaded to an information security system, the information security system is synchronized to a doctor end of the information security system for being inquired by a doctor, and a prescription interface inquired by the doctor is shown in FIG. 20; the signed prescription Information may also be synchronized to the HIS (Hospital Information System) inside the Hospital.

Optionally, the internet hospital system may modify the electronic file (e.g., a hospital order or prescription) in response to the received electronic file modification instruction.

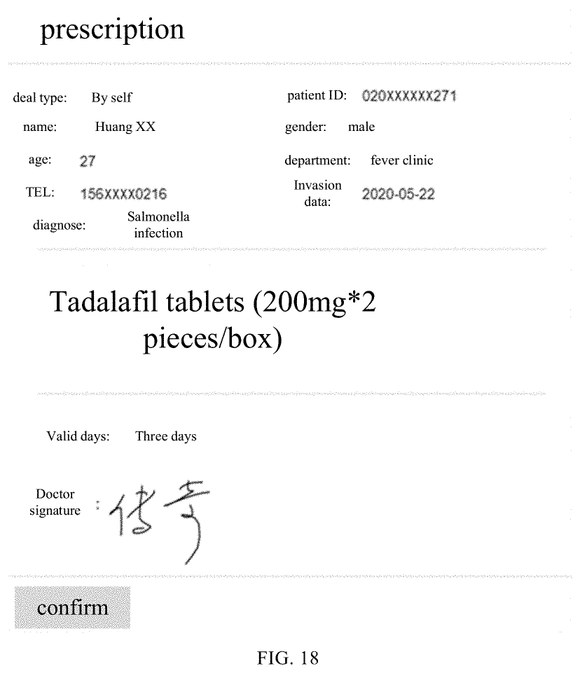

It should be noted that the numerical values of the parameters such as the oral specification, the dosage, the amount of money, the expiration date and the like in FIGS. 17 to 20 of the present disclosure are only examples, and do not represent actual numerical values; the doctor end and the patient end in the embodiment of the present disclosure may be any one of a PC (Personal Computer) end, an APP (application program) end, and an applet end.

Optionally, the medical personnel information management method provided in the embodiment of the present disclosure further includes:

in response to a login request of any medical personnel for logging in the medical personnel management system, and displaying an authorization interface for calling the electronic signature or not; in response to the instruction to grant authorization, the login request is granted and authorization information is sent to the information security system.

In one example, if the authorization interface for calling the electronic signature is authorized as shown in FIG. 17, the user can scan the two-dimensional code shown in FIG. 17 to authorize the electronic signature.

When medical personnel logs in the internet hospital system, the risk of logging in a different place or multiple devices may exist, the login request is verified in a mode of electronic signature authorization from the aspect of safety, and when the medical personnel logs in again after logging out of the internet hospital system, the authentication authorization of the electronic signature is needed, so that the safety of logging in the different place and the multiple devices can be effectively improved, the authorization of the electronic signature can be realized by a user through a daily mobile phone or a tablet personal computer, and the operation convenience can be improved.

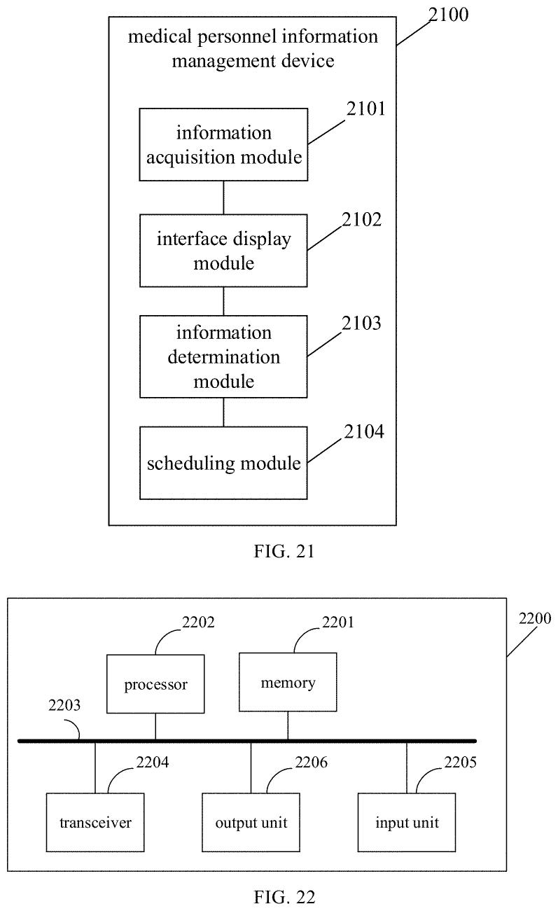

Based on the same inventive concept, a medical personnel information management device provided in the embodiment of the present disclosure can be applied to a medical personnel management system (internet hospital system), and as shown in FIG. 21, the medical personnel information management device 2100 includes: the system includes an information acquisition module 2101, an interface display module 2102, an information determination module 2103 and a scheduling module 1204.

The information obtaining module 2101 is configured to determine at least one medical personnel when a selection instruction for the at least one medical personnel is received.

The interface display module 2102 is used for in response to the received scheduling command and displaying a scheduling data configuration interface;

the information determining module 2103 is used for in response to the received scheduling instruction, displaying a scheduling data configuration interface, and determining the to-be-scheduled date and whether to use a scheduling template for scheduling;

the scheduling module 2104 is used for calling the scheduling template to perform scheduling on each medical personnel when the scheduling template is determined to be used for scheduling, so as to obtain scheduling data; when the scheduling template is determined not to be used for scheduling, in response to a scheduling data configuration instruction, configuring a plurality of to-be-scheduled time periods in the dates to be scheduled and the number of available medical personnel in association with each to-be-scheduled time period for each medical personnel on a scheduling data configuration interface, and obtaining scheduling data; the scheduling template includes to-be-scheduled time periods in the day to be scheduled and the number of available doctors associated with each to-be-scheduled time period.

Optionally, the scheduling module 2104 is further for: determining whether to perform batch scheduling; when the batch scheduling is determined, a plurality of target dates which take the to-be-scheduled dates as the starting points and are within the preset batch scheduling date range are determined, and the scheduling data are applied to each target date.

Optionally, the scheduling module 2104 is specifically configured to: and acquiring at least one scheduling date strategy, and determining a plurality of dates which accord with the batch scheduling date range and each scheduling date strategy by taking the to-be-scheduled date as a starting point to be used as a plurality of target dates.

The at least one scheduling date strategy includes at least one of: date excepting weekends, date excepting holidays, each date in a specified range of consecutive dates reserved, odd dates reserved, even dates reserved.

Optionally, the medical personnel information management device provided in the embodiment of the present disclosure further includes: and a template configuration module.

The template configuration module is used for: in response to a template configuration instruction, and configuring at least one template name in a template configuration interface; and for each template name, configuring a plurality of to-be-scheduled time periods and the number of visitors who can receive a doctor and have an association relationship with each to-be-scheduled time period, and storing the number of visitors.

Optionally, the medical personnel information management device that this application implementation provided still includes: the system includes a dimension determining module, a scheduling data acquiring module and an information displaying module.

The dimension determination module is to: determining dimension information for displaying the scheduling data, and determining a date to be displayed.

The scheduling data acquisition module is used for: and acquiring the scheduling data of the medical personnel regularly according to the dimension information and the date to be displayed.

The information display module is used for: and displaying corresponding scheduling data.

Optionally, the information display module is further configured to: and determining and displaying the current scheduling occupancy rate according to the used scheduling data of the current date and all the scheduling data of the current date.

Optionally, the information display module is further configured to: in response to an information query instruction for the current scheduling time period, and displaying at least one of reservation information, used scheduling data and to-be-used scheduling data of the current scheduling time period; the used scheduling data is actually used data before the current time in the scheduling data of the current date, and the to-be-used scheduling data is data except the used scheduling data in all the scheduling data of the current date.

Optionally, the medical personnel information management device that this application implementation provided still includes: the device includes an overtime judging module and an early warning module.

The timeout determining module is configured to: determining whether the work of each medical personnel is overtime according to the scheduling time period to which the used scheduling data of each medical personnel belongs and the time-position relation of the current time; the used scheduling data is data which is actually used before the current time in the scheduling data of the current date. The early warning module is used for: and when the work of the medical personnel is overtime, first early warning information is sent to a display terminal of the medical personnel.

Optionally, the timeout determining module is further configured to: when the work of the medical personnel is not overtime, whether the work of the medical personnel is overtime is predicted according to the proportion relation between the data belonging to the current scheduling time period in the used scheduling data and the scheduling data of the current scheduling time period. The early warning module is also used for: and when the working time of the medical personnel is predicted to be out, second early warning information is sent to a display terminal of the medical personnel.

The medical personnel information management device 2100 in this embodiment can execute any one of the medical personnel information management methods provided in the embodiments of the present disclosure, and the implementation principles thereof are similar, and details that are not shown in detail in this embodiment can refer to the foregoing embodiments, and are not described herein again.

The modules may be implemented based on software, hardware, firmware, and the like, and are not limited herein.

Based on the same inventive concept, an embodiment of the present disclosure provides a medical personnel information management device, including: the storage is electrically connected to the processor.

The memory has stored thereon a computer program that is executed by the processor to perform:

when a selection instruction for at least one medical personnel is received, determining at least one medical personnel;

in response to the received scheduling instruction, and displaying a scheduling data configuration interface;

determining the to-be-scheduled date and whether a scheduling template is used for scheduling;

when the scheduling template is determined to be used for scheduling, the scheduling template is called to perform scheduling for each medical personnel, and scheduling data are obtained; when the scheduling template is determined not to be used for scheduling, in response to a scheduling data configuration instruction, and configuring a plurality of to-be-scheduled time periods and the number of available consultants in association with each to-be-scheduled time period in the to-be-scheduled date for each medical personnel on the scheduling data configuration interface to obtain scheduling data;

the scheduling template includes the to-be-scheduled time periods in the day to be scheduled and the number of available doctors which are associated with each to-be-scheduled time period.

Optionally, the scheduling template is configured in the following manner:

in response to a template configuration instruction, and configuring at least one template name in a template configuration interface;

and for each template name, configuring a plurality of to-be-scheduled time periods and the number of visitors who can receive a doctor and have an association relationship with each to-be-scheduled time period, and storing the number of visitors.

Optionally, the computer program is executed by the processor to implement:

determining whether to perform batch scheduling;

when the batch scheduling is determined, a plurality of target dates which take the to-be-scheduled dates as starting points and are within a preset batch scheduling date range are determined, and the scheduling data are applied to each target date.

Optionally, the computer program is executed by the processor to implement:

acquiring at least one scheduling date strategy, determining a plurality of dates which accord with the batch scheduling date range and each scheduling date strategy by taking the to-be-scheduled date as a starting point, and taking the dates as a plurality of target dates;

the at least one scheduling date strategy includes at least one of: date excepting weekends, date excepting holidays, each date in a specified range of consecutive dates reserved, odd dates reserved, even dates reserved.

Optionally, the computer program is executed by the processor to implement:

determining dimension information for displaying the scheduling data, and determining a date to be displayed;

acquiring the scheduling data of the medical personnel regularly according to the dimension information and the date to be displayed;

and displaying the corresponding scheduling data.

Optionally, the computer program is executed by the processor to implement:

determining and displaying the current scheduling occupancy rate according to the used scheduling data of the current date and all the scheduling data of the current date; the used scheduling data are data which are actually used before the current time in the scheduling data of the current date.

Optionally, the computer program is executed by the processor to implement:

determining whether the work of each medical personnel is overtime according to the scheduling time period to which the used scheduling data of each medical personnel belongs and the time-position relation of the current time; the used scheduling data is actually used data before the current time in the scheduling data of the current date;

and if the work of the medical personnel is overtime, sending first early warning information to a display terminal of the medical personnel.

Optionally, the computer program is executed by the processor to implement:

if the work of the medical personnel is not overtime, predicting whether the work of the medical personnel is overtime according to the proportional relation between the data belonging to the current scheduling time period in the used scheduling data and the scheduling data of the current scheduling time period;

and if the working time of the medical personnel is predicted to be overtime, sending second early warning information to a display terminal of the medical personnel.

Optionally, the computer program is executed by the processor to implement:

in response to an information query instruction for the current scheduling time period, and displaying at least one of reservation information, used scheduling data and to-be-used scheduling data of the current scheduling time period; the used scheduling data is actually used data before the current time in the scheduling data of the current date, and the to-be-used scheduling data is data except the used scheduling data in all the scheduling data of the current date.

Optionally, the computer program is executed by the processor to implement:

sending basic information of each medical personnel to an information security system, and receiving a unique identification of each medical personnel fed back by the information security system based on the basic information;

in response to an electronic signature calling request of any medical personnel, calling an interface associated with the unique identification of the medical personnel to obtain the requested electronic signature.

Optionally, the computer program is executed by the processor to implement:

in response to a login request of any medical personnel for logging in the medical personnel management system, and displaying an authorization interface for calling the electronic signature or not;

in response to the instruction to grant authorization, the login request is granted and authorization information is sent to the information security system.

Optionally, the calling, in response to an electronic signature calling request of any medical personnel, an interface associated with the unique identifier of the medical personnel to obtain the requested electronic signature includes:

when an electronic signature calling request of any medical personnel is received, the electronic signature calling request is sent to the information security system;

in response to an agreeing calling instruction fed back by the information security system, and calling an interface associated with the unique identifier of the medical personnel to acquire an electronic signature of the medical personnel; the consent call instruction is generated by the information security system upon verifying that the electronic signature is authorized;

in response to a call rejection instruction fed back by the information security system, displaying an authorization interface for judging whether to authorize to call the electronic signature, in response to an authorization approval instruction, calling an interface associated with the unique identifier of the medical personnel to acquire the electronic signature of the medical personnel, and uploading authorization information to the information security system; the rejection call instruction is generated by the information security system when the electronic signature is verified to be unauthorized.

It will be appreciated by those skilled in the art that the medical personnel information management device provided in the embodiments of the present disclosure may be specially designed and manufactured for the required purposes, or may include a known device in a general-purpose computer. These devices have stored within them computer programs that are selectively activated or reconfigured. Such a computer program may be stored in a device (e.g., computer) readable medium or in any type of medium suitable for storing electronic instructions and respectively coupled to a bus.

The present disclosure provides, in an alternative embodiment, a medical personnel information management device, as shown in FIG. 22, the medical personnel information management device 2200 including: the memory 2201 and the processor 2202, the memory 2201 and the processor 2202 being electrically connected, such as by a bus 2203.

Optionally, the memory 2201 is used for storing application program codes for implementing the schemes of the present disclosure and the execution of which is controlled by the processor 2202. The processor 1302 is configured to execute the application program code stored in the memory 1301, so as to implement any one of the medical personnel information management methods provided in the embodiments of the present disclosure.

The Memory 2201 may be a ROM (Read-Only Memory) or other type of static storage device that can store static information and instructions, a RAM (Random Access Memory) or other type of dynamic storage device that can store information and instructions, an EEPROM (Electrically Erasable Programmable Read Only Memory), a CD-ROM (Compact Disc Read-Only Memory) or other optical disk storage, optical disk storage (including Compact Disc, laser Disc, optical Disc, digital versatile Disc, blu-ray Disc, etc.), a magnetic disk storage medium or other magnetic storage device, or any other medium that can be used to carry or store desired program code in the form of instructions or data structures and that can be accessed by a computer, but is not limited thereto.

The Processor 2202 may be a CPU (Central Processing Unit), a general purpose Processor, a DSP (Digital Signal Processor), an ASIC (Application Specific Integrated Circuit), an FPGA (Field-Programmable Gate Array), or other Programmable logic device, transistor logic, hardware components, or any combination thereof. Which may implement or execute the various illustrative logical blocks, modules, and circuits described in connection with the disclosure herein. The processor 2202 can also be a combination that performs a computing function, e.g., including one or more microprocessors, a combination of DSPs and microprocessors, or the like.

Bus 2203 may include a path that transfers information between the above components. The bus may be a PCI (Peripheral Component Interconnect) bus or an EISA (Extended Industry Standard Architecture) bus. The bus may be divided into an address bus, a data bus, a control bus, etc. For ease of illustration, only one thick line is shown in FIG. 22, but that does not indicate only one bus or one type of bus.

Optionally, the medical personnel information management device 2200 may also include a transceiver 2204. The transceiver 2204 may be used for reception and transmission of signals. The transceiver 2204 may allow the medical personnel information management device 2200 to communicate wirelessly or wiredly with other devices to exchange data. It should be noted that the transceiver 2204 is not limited to one in practical application.

Optionally, the medical personnel information management device 2200 may further include an input unit 2205. The input unit 2205 may be used to receive input numeric, character, image and/or sound information or to generate key signal inputs related to user settings and function control of the electronic device 2200. The input unit 2205 may include, but is not limited to, one or more of a touch screen, a physical keyboard, function keys (such as volume control keys, switch keys, etc.), a trackball, a mouse, a joystick, a camera, a microphone, and the like.

Optionally, the medical personnel information management device 2200 may further include an output unit 2206. The output unit 2206 may be used to output or present information processed by the processor 2202. The output unit 2206 may include, but is not limited to, one or more of a display device, a speaker, a vibration device, and the like.

While FIG. 22 illustrates a medical personnel information management device 2200 with various means, it is to be understood that not all illustrated means are required to be implemented or provided. More or fewer devices may be alternatively implemented or provided.

Although the embodiments of the present disclosure provide some embodiments or method operation instruction steps shown in the figures, more or less operation instruction steps can be included in the method based on the conventional or non-creative work. In steps where no necessary causal relationship exists logically, the order of execution of these steps is not limited to the order of execution provided by the embodiments of the present disclosure.

Based on the same inventive concept, the present disclosure provides a computer-readable storage medium, on which a computer program is stored, and when the computer program is executed by a processor, the computer program implements any one of the medical personnel information management methods provided by the embodiments of the present disclosure.

The computer readable medium can include, but is not limited to, any type of disk including floppy disks, hard disks, optical disks, CD-ROMs, and magnetic-optical disks, ROMs, RAMs, EPROMs (Erasable Programmable Read-Only memories), EEPROMs, flash memories, magnetic cards, or fiber optic cards. That is, readable media includes any medium that stores or transmits information in a form readable by a device (e.g., a computer).

The embodiment of the present disclosure provides a computer-readable storage medium, which is applicable to any of the above employee information management methods, and details are not described herein again.

By applying the technical scheme of the embodiment of the application, the following beneficial effects can be at least realized:

1) compared with the traditional scheduling mode, the method and the device have the advantages that the scheduling can be automatically performed on each medical personnel based on the determined basic information of the medical personnel and the to-be-scheduled date, manual scheduling is not needed, each medical personnel is scheduled respectively, operation is simpler and more efficient, the scheduling efficiency can be effectively improved, and the scheduling requirement of a hospital is met.

2) According to the method and the device, the scheduling template can be configured in advance, and when the scheduling template is required to be used for scheduling, the data of the scheduling template can be directly called for scheduling, so that the scheduling efficiency is further improved; meanwhile, the embodiment of the application can allocate a plurality of to-be-scheduled time periods to a certain date for scheduling in time periods, so that the granularity of scheduling can be increased, the scheduling is more refined, the scheduling utilization rate is improved, and the requirement of surge under emergency (such as epidemic situation) can be better met.

3) According to the embodiment of the application, batch scheduling of a plurality of dates to be scheduled can be realized, and a single medical personnel, a plurality of medical personnels, and all medical personnels in the whole department or department can be scheduled in a batch scheduling mode, so that the scheduling efficiency is further improved; based on a preset scheduling date strategy, the to-be-scheduled dates needing batch scheduling can be quickly determined so as to meet the scheduling requirements of different enterprises and public institutions or different periods.

4) The scheduling data after scheduling is finished can be dynamically acquired and displayed so as to observe the scheduling condition; the scheduling data can be displayed according to the dimension information, the required single-dimension or multi-dimension data display can be realized, the displayed data is more targeted, and data support is provided for optimizing subsequent scheduling.

5) Based on the scheduling data of the current date and the used scheduling data, the scheduling occupancy rate and the overtime condition of the work of medical personnel can be determined, the scheduling occupancy rate is displayed, early warning is carried out based on the overtime condition, the actual use condition of the scheduling data is convenient to know in time, staff are reminded to take corresponding measures to deal with the overtime condition or the overtime condition is predicted in an early warning mode, the efficiency of overall work scheduling is improved, and the waiting of visitors (such as patients who go to hospitals for medical treatment) is reduced.

6) According to the embodiment of the application, the related information of medical personnel can be synchronized with the information security system, so that the dual storage and backup of the information are realized, the paperless management of the information is realized, the retrospective query of data is facilitated, and the basic data is provided for the association analysis and the mining of big data; the method includes the steps that the unique identification is distributed based on the basic information of each medical personnel, the association relation between the medical personnel and the interface can be established, information in the information security system is called based on the association relation, the information barrier between the medical personnel management system and the information security system can be overcome, seamless butt joint between the medical personnel management system and the information security system is achieved, and meanwhile the information calling security is improved.

7) Compared with the existing U-key login and identity authentication based information calling mode, the mode of the embodiment of the application does not need to use a hardware authentication device, and is more convenient to use on the basis of improving the login authentication security because the problems of loss and damage do not exist and a special CA environment does not need to be installed.

8) When an electronic signature calling request is received, the authorization condition of the electronic signature is firstly verified, the electronic signature is agreed to be called only under the condition that the electronic signature is authorized, otherwise, the authorization of the electronic signature is required to be carried out again, the use safety of the electronic signature is guaranteed while automatic signature is realized, the working efficiency and the signature quality of medical personnels are improved, the teleworking mode is optimized, and the working time cost of the medical personnels is saved to the greatest extent.

9) The embodiment of the application has wide application range, is suitable for hospital scenes, optimizes the treatment mode of remote diagnosis and treatment plus electronic prescription, is convenient for doctors to perform on-line diagnosis, make prescriptions, check, examine and the like, and enhances the on-line inquiry function of the Internet hospital so as to meet the demand of on-line inquiry of sudden public health incidents, thereby serving as powerful supplement and support for entity hospitals.

10) The embodiment of the application can also be applied to application scenes of various enterprises and public institutions such as banks, social security offices and the like, including units with visitor reception tasks.

Those of skill in the art will understand that various operations, methods, steps in the flow, measures, schemes discussed in this application can be alternated, modified, combined, or deleted. Further, various operations, methods, steps, measures, schemes in the various processes, methods, procedures that have been discussed in this application may be alternated, modified, rearranged, decomposed, combined, or eliminated. Further, the steps, measures, and schemes in the various operations, methods, and flows disclosed in the present disclosure in the prior art can also be alternated, modified, rearranged, decomposed, combined, or deleted.

In the description of the present disclosure, it is to be understood that the terms “first”, “second” and “first” are used for descriptive purposes only and are not to be construed as indicating or implying relative importance or as implying that the number of indicated technical features is indicated. Thus, a feature defined as “first” or “second” may explicitly or implicitly include one or more of that feature. In the description of the present disclosure, the meaning of “a plurality” is two or more unless otherwise specified.

It should be understood that, although the steps in the flowcharts of the figures are shown in order as indicated by the arrows, the steps are not necessarily performed in order as indicated by the arrows. The steps are not performed in the exact order shown and may be performed in other orders unless otherwise indicated herein. Moreover, at least a portion of the steps in the flow chart of the figure may include multiple sub-steps or multiple stages, which are not necessarily performed at the same time, but may be performed at different times, and the order of execution is not necessarily sequential, but may be performed alternately or alternately with other steps or at least a portion of the sub-steps or stages of other steps.

The foregoing is only a few embodiments of the present disclosure and it should be noted that those skilled in the art can make various improvements and modifications without departing from the principle of the present disclosure, and that these improvements and modifications should also be considered as the protection scope of the present disclosure.

What is claimed is:

1. A medical personnel information management method, comprising:
when a selection instruction for at least one medical personnel is received, determining at least one medical personnel;
displaying a scheduling data configuration interface in response to a received scheduling instruction;
determining a to-be-scheduled date and whether a scheduling template is used for scheduling;
when the scheduling template is determined to be used for scheduling, calling the scheduling template to perform schedule each medical personnel to obtain scheduling data, wherein the scheduling template comprises to-be-scheduled time periods and an acceptable number of visitors associated with each to-be-scheduled time period in the to-be-scheduled date;
when the scheduling template is determined not to be used for scheduling, in response to a scheduling data configuration instruction, configuring a plurality of to-be-scheduled time periods and an acceptable number of visitors associated with each to-be-scheduled time period in the to-be-scheduled date for each medical personnel on the scheduling data configuration interface, to obtain the scheduling data.

2. The medical personnel information management method according to claim 1, wherein the scheduling template is configured by:
configuring at least one template name in a template configuration interface in response to a template configuration instruction; and
for each template name, configuring a plurality of to-be-scheduled time periods and the acceptable number of visitors associated with each to-be-scheduled time period, and storing the acceptable number of visitors.

3. The medical personnel information management method according to claim 1, further comprising:
determining whether to perform a batch scheduling;
when the batch scheduling is determined to be performed, determining a plurality of target dates which take the to-be-scheduled dates as starting points and are within a preset batch scheduling date range, and applying the scheduling data to each target date.

4. The medical personnel information management method according to claim 3, wherein the determining the plurality of target dates which take the to-be-scheduled dates as the starting points and are within the preset batch scheduling date range comprises:
acquiring at least one scheduling date strategy, determining a plurality of dates which accord with the batch scheduling date range and each scheduling date strategy by taking the to-be-scheduled dates as the starting points, and taking the dates as a plurality of target dates;
the at least one scheduling date strategy comprises at least one of: date excepting weekends, date excepting holidays, each date in a specified range of consecutive dates reserved, odd dates reserved, even dates reserved.

5. The medical personnel information management method according to claim 1, further comprising:
determining dimension information for displaying the scheduling data, and determining a date to be displayed;
acquiring the scheduling data of the medical personnel regularly according to the dimension information and the date to be displayed; and
displaying the corresponding scheduling data.

6. The medical personnel information management method according to claim 5, further comprising:
determining and displaying a current scheduling occupancy rate according to the used scheduling data of the current date and all the scheduling data of the current date; the used scheduling data are data which are actually used before the current time in the scheduling data of the current date.

7. The medical personnel information management method according to claim 5, further comprising:
determining whether the work of each medical personnel is overtime according to a time-position relation between a scheduling time period to which the used scheduling data of each medical personnel belongs and the current time; the used scheduling data is an actually used data before the current time in the scheduling data of the current date;
if the work of the medical personnel is overtime, sending first early warning information to a display terminal of the medical personnel.

8. The medical personnel information management method according to claim 7, further comprising:
if the work of the medical personnel is not overtime, predicting whether the work of the medical personnel is overtime according to a proportional relation between the data belonging to the current scheduling time period in the used scheduling data and the scheduling data of the current scheduling time period;
if the working time of the medical personnel is predicted to be overtime, sending second early warning information to the display terminal of the medical personnel.

9. The medical personnel information management method according to claim 5, further comprising:
in response to an information query instruction for the current scheduling time period, displaying at least one of reservation information, used scheduling data and to-be-used scheduling data of the current scheduling time period; the used scheduling data is actually used data before the current time in the scheduling data of the current date, and the to-be-used scheduling data is data except the used scheduling data in all the scheduling data of the current date.

10. The medical personnel information management method according to claim 1, further comprising:
sending basic information of each medical personnel to an information security system, and receiving a unique identification of each medical personnel fed back by the information security system based on the basic information;
in response to an electronic signature calling request of any one of medical personnel, calling an interface associated with the unique identification of the medical personnel to obtain a requested electronic signature.

11. The medical personnel information management method according to claim 10, further comprising:
in response to a login request of any medical personnel for logging in a medical personnel management system, displaying an authorization interface displaying whether the calling of electronic signature is allowed;
in response to an instruction of allowing the calling of electronic signature, allowing the login request and sending authorization information to the information security system.

12. The medical personnel information management method according to claim 10, wherein the in response to an electronic signature calling request of any one of medical personnel, calling the interface associated with the unique identification of the medical personnel to obtain the requested electronic signature comprises:
when an electronic signature calling request of any medical personnel is received, sending the electronic signature calling request to the information security system;
in response to an instruction of allowing the calling of electronic signature fed back by the information security system, calling the interface associated with the unique identifier of the medical personnel to acquire an electronic signature of the medical personnel; the instruction of allowing the calling of electronic signature is generated by the information security system upon verifying that the electronic signature is authorized;
in response to a call rejection instruction fed back by the information security system, displaying an authorization interface for judging whether to authorize to call the electronic signature, in response to an authorization approval instruction, calling the interface associated with the unique identifier of the medical personnel to acquire the electronic signature of the medical personnel, and uploading authorization information to the information security system; the rejection call instruction is generated by the information security system when the electronic signature is verified to be unauthorized.

13. A medical personnel information management device, comprising: a memory, a processor electrically connected to the memory, wherein a computer program is stored in the memory, and the processor is configured to execute the computer program to:
when a selection instruction for at least one medical personnel is received, determine at least one medical personnel;
display a scheduling data configuration interface in response to a received scheduling instruction;
determine a to-be-scheduled date and whether a scheduling template is used for scheduling;
when the scheduling template is determined to be used for scheduling, call the scheduling template to perform schedule each medical personnel to obtain scheduling data, wherein the scheduling template comprises to-be-scheduled time periods and an acceptable number of visitors associated with each to-be-scheduled time period in the to-be-scheduled date;
when the scheduling template is determined not to be used for scheduling, in response to a scheduling data configuration instruction, configure a plurality of to-be-scheduled time periods and an acceptable number of visitors associated with each to-be-scheduled time period in the to-be-scheduled date for each medical personnel on the scheduling data configuration interface, to obtain the scheduling data.

14. The medical personnel information management device according to claim 13, wherein the scheduling template is configured by:
configuring at least one template name in a template configuration interface in response to a template configuration instruction; and
for each template name, configuring a plurality of to-be-scheduled time periods and the acceptable number of visitors associated with each to-be-scheduled time period, and storing the acceptable number of visitors.

15. The medical personnel information management device according to claim 13, wherein the processor is configured to execute the computer program to:
determine whether to perform a batch scheduling;
when the batch scheduling is determined to be performed, determine a plurality of target dates which take the to-be-scheduled dates as starting points and are within a preset batch scheduling date range, and apply the scheduling data to each target date.

16. The medical personnel information management device according to claim 15, wherein the processor is configured to execute the computer program to:
acquire at least one scheduling date strategy, determining a plurality of dates which accord with the batch scheduling date range and each scheduling date strategy by taking the to-be-scheduled dates as the starting points, and take the dates as a plurality of target dates;
the at least one scheduling date strategy comprises at least one of: date excepting weekends, date excepting holidays, each date in a specified range of consecutive dates reserved, odd dates reserved, even dates reserved.

17. The medical personnel information management device according to claim 13, wherein the processor is configured to execute the computer program to:
determine dimension information for displaying the scheduling data, and determining a date to be displayed;
acquire the scheduling data of the medical personnel regularly according to the dimension information and the date to be displayed; and
display the corresponding scheduling data.

18. The medical personnel information management device according to claim 17, wherein the processor is configured to execute the computer program to:
determine and display a current scheduling occupancy rate according to the used scheduling data of the current date and all the scheduling data of the current date;
the used scheduling data are data which are actually used before the current time in the scheduling data of the current date.

19. The medical personnel information management device according to claim 17, wherein the processor is configured to execute the computer program to:
determine whether the work of each medical personnel is overtime according to a time-position relation between a scheduling time period to which the used scheduling data of each medical personnel belongs and the current time; the used scheduling data is an actually used data before the current time in the scheduling data of the current date;
if the work of the medical personnel is overtime, send first early warning information to a display terminal of the medical personnel.

20. A non-transitory memory computer-readable storage medium, storing a computer program, where the computer program is executed by a processor to:
when a selection instruction for at least one medical personnel is received, determine at least one medical personnel;
display a scheduling data configuration interface in response to a received scheduling instruction;
determine a to-be-scheduled date and whether a scheduling template is used for scheduling;
when the scheduling template is determined to be used for scheduling, call the scheduling template to perform schedule each medical personnel to obtain scheduling data, wherein the scheduling template comprises to-be-scheduled time periods and an acceptable number of visitors associated with each to-be-scheduled time period in the to-be-scheduled date;
when the scheduling template is determined not to be used for scheduling, in response to a scheduling data configuration instruction, configure a plurality of to-be-scheduled time periods and an acceptable number of visitors associated with each to-be-scheduled time period in the to-be-scheduled date for each medical personnel on the scheduling data configuration interface, to obtain the scheduling data.
